(19) United States
(12) Patent Application Publication
(10) Pub. No.: US 2020/0121378 A1
(43) Pub. Date: Apr. 23, 2020

(54) METHOD OF DETERMINING SETTING PARAMETER FOR TEMPERATURE CONTROL OF SURGICAL SYSTEM, HANDPIECE OF SURGICAL SYSTEM, AND SURGICAL SYSTEM

(71) Applicant: Olympus Corporation, Tokyo (JP)

(72) Inventor: Toshifumi Katsuragi, Tokyo (JP)

(73) Assignee: Olympus Corporation, Tokyo (JP)

(21) Appl. No.: 16/718,468

(22) Filed: Dec. 18, 2019

Related U.S. Application Data
(63) Continuation of application No. PCT/JP2017/023776, filed on Jun. 28, 2017.

(51) Int. Cl. A61B 18/10

(52) U.S. Cl. CPC A61B 18/10; A61B 2018/0063

(57) ABSTRACT

The disclosed technology is directed to a handpiece for a surgical system. The handpiece comprises a heater, a first detection circuit configured to measure a voltage and a current supplied to the heater, and a memory. The memory stores detection characteristics representing a correlation between a sensor value of the first detection circuit and the at least one of the voltage value and the current value. The correlation is used for determination of one of the voltage value and the current value is applied to the heater as obtained using a second detection circuit having a higher detection accuracy than the first detection circuit. A control parameter is used to control a temperature of the heater and is determined based on temperature changes of the heater while performing a test control that supplies electric power to the heater to control the temperature of the heater in a predetermined manner.

This application is a continuation application of PCT Application No. PCT/JP2017/023776 filed on Jun. 28, 2017, which is hereby incorporated by reference in its entirety.

### TECHNICAL FIELD

The disclosed technology relates to a method of manufacturing a handpiece for a surgical system, a method of setting characteristics of the handpiece for the surgical system, the handpiece for the surgical system, and the surgical system.

### DESCRIPTION OF THE RELATED ART

Known surgical systems include those which grasp a body tissue as a target treatment site by a grasping portion including a heater and treat the body tissue with heat generated by the heater. For example, JP 2007-37845A discloses a surgical system that can apply heat, which has been generated by a heater, to a body tissue grasped by a grasping portion. In this surgical system, information regarding individual differences in characteristics of a handpiece for the surgical system is stored in a memory included in the handpiece. A control device reads the information stored in the memory, and controls the operation of the handpiece by using the information. The control can be realized with higher accuracy by performing it with individual differences of a handpiece taken into consideration.

For accurate control of operation of a handpiece, appropriate information regarding individual differences of the handpiece is needed.

### BRIEF SUMMARY OF EMBODIMENTS

The disclosed technology has been made in view of the foregoing.

The disclosed technology is directed to a method of determining setting parameters for temperature control of a handpiece used in a surgical system. The handpiece includes a heater, a first detection circuit configured to measure a voltage and a current to be supplied to the heater, and a memory. The method comprises obtaining a detection characteristic representing a correlation between a sensor value of the first detection circuit and the at least one of the voltage value and the current value. The correlation is used for determination of the at least one of the voltage value and the current value being applied to the heater and based on a result measured using a second detection circuit having a higher detection accuracy than the first detection; storing the detection characteristic in the memory; obtaining temperature changes of the heater while performing a test control that supplies electric power to the heater to control a temperature of the heater in a predetermined manner; determining, based on the temperature changes, a control parameter which is to be used to control the temperature of the heater; and storing the control parameter in the memory.

Another aspect of the disclosed technology is directed to a handpiece for a surgical system. The handpiece comprises a heater; a first detection circuit configured to measure a voltage and a current to be supplied to the heater; and a memory. The memory stores detection characteristics representing a correlation between a sensor value of the first detection circuit and the at least one of the voltage value and the current value. The correlation being used for determination of the at least one of the voltage value and the current value being applied to the heater as obtained based on results measured using a second detection circuit having a higher detection accuracy than the first detection circuit. A control parameter which is to be used to control a temperature of the heater and is determined based on temperature changes of the heater while performing a test control that supplies electric power to the heater to control the temperature of the heater in a predetermined manner.

A further aspect of the disclosed technology is directed to a surgical system comprises a handpiece having a heater; a first detection circuit configured to measure a voltage and a current to be supplied to the heater, and a memory. The memory stores detection characteristics representing a correlation between a sensor value of the first detection circuit and the at least one of the voltage value and the current value. The correlation is used for determination of the at least one of the voltage value and the current value being applied to the heater as obtained based on results measured using a second detection circuit having a higher detection accuracy than the first detection circuit. A control parameter which is to be used to control a temperature of the heater and is determined based on temperature changes of the heater while performing a test control that supplies electric power to the heater to control the temperature of the heater in a predetermined manner. A power source device configured to supply electric power to the heater of the handpiece. The power source device is configured to obtain the detection characteristics and the control parameter from the memory of the handpiece and control the electric power which is to be supplied to the heater, based on a sensor value obtained by the first detection circuit, the detection characteristics, and the control parameter.

### BRIEF DESCRIPTION OF THE DRAWINGS

The technology disclosed herein, in accordance with one or more various embodiments, is described in detail with reference to the following figures. The drawings are provided for purposes of illustration only and merely depict typical or example embodiments of the disclosed technology. These drawings are provided to facilitate the reader's understanding of the disclosed technology and shall not be considered limiting of the breadth, scope, or applicability thereof. It should be noted that for clarity and ease of illustration these drawings are not necessarily made to scale.

FIG. 1A is a block diagram illustrating an overview of configuration examples of a surgical system and a setting system according to an embodiment of the disclosed technology.

FIG. 1B is a block diagram illustrating an overview of configuration examples of the surgical system and the setting system upon performing detection characteristic setting processing according to the embodiment.

FIG. 1C is a block diagram illustrating an overview of configuration examples of the surgical system and the setting system upon performing control parameter setting processing according to the embodiment.

FIG. 2 is a view illustrating an overview of a configuration example of the surgical system according to the embodiment.

FIG. 3 is a flow chart for making an explanation about an outline of an example of a method of setting the surgical system according to the embodiment.

FIG. 4 is a flow chart illustrating an outline of an example of the detection characteristic setting processing according to the embodiment.

FIG. 5 is a diagram for making an explanation about an example of detection characteristic data of a voltage value at a first detection circuit according to the embodiment.

FIG. 6 is a diagram for making an explanation about an example of detection characteristic data of a current value at the first detection circuit according to the embodiment.

FIG. 7A is a flow chart illustrating an outline of an example of the control parameter setting processing according to the embodiment.

FIG. 7B is an additional flow chart illustrating the outline of the example of the control parameter setting processing according to the embodiment.

FIG. 8 is a schematic diagram for making an explanation about an example of test control.

FIG. 9 is a schematic diagram for making an explanation about an example of temperature changes of a heater during test control.

FIG. 10 is a schematic diagram for making an explanation about another example of temperature changes of the heater during the test control.

FIG. 11 is a schematic diagram for making an explanation about a further example of temperature changes of the heater during the test control.

FIG. 12 is a schematic diagram for making an explanation about a still further example of temperature changes of the heater during the test control.

FIG. 13 is a schematic diagram for making an explanation about a yet further example of temperature changes of the heater during the test control.

FIG. 14A is a block diagram illustrating an overview of configuration examples of a surgical system and a setting system according to a first modification upon performing detection characteristic setting processing.

FIG. 14B is a block diagram illustrating an overview of configuration examples of the surgical system and the setting system according to the first modification upon performing control parameter setting processing.

FIG. 15A is a block diagram illustrating an overview of configuration examples of a surgical system and a setting system according to a second modification.

FIG. 15B is a block diagram illustrating an overview of configuration examples of the surgical system and the setting system according to the second modification upon performing detection characteristic setting processing.

FIG. 15C is a block diagram illustrating an overview of configuration examples of the surgical system and the setting system according to the second modification upon performing control parameter setting processing.

### DETAILED DESCRIPTION OF THE EMBODIMENTS

In the following description, various embodiments of the technology will be described. For purposes of explanation, specific configurations and details are set forth in order to provide a thorough understanding of the embodiments. However, it will also be apparent to one skilled in the art that the technology disclosed herein may be practiced without the specific details. Furthermore, well-known features may be omitted or simplified in order not to obscure the embodiment being described.

The disclosed technology has as objects thereof the provision of a method of manufacturing a handpiece for a surgical system, the handpiece enabling accurate control, a method of setting characteristics of the handpiece for the surgical system, the handpiece for the surgical system, and the surgical system.

### System Configuration

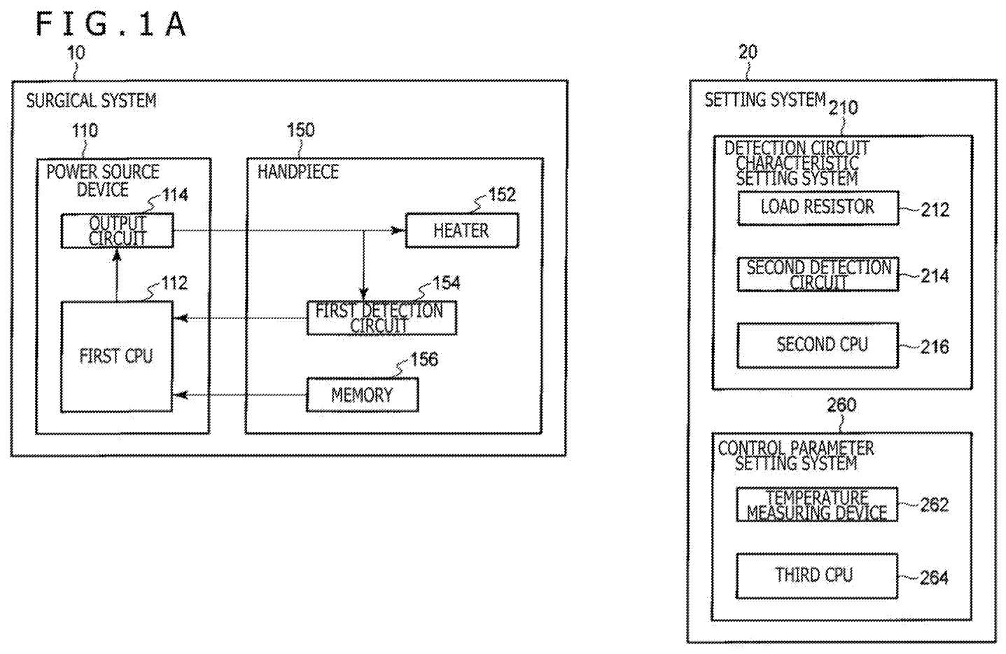

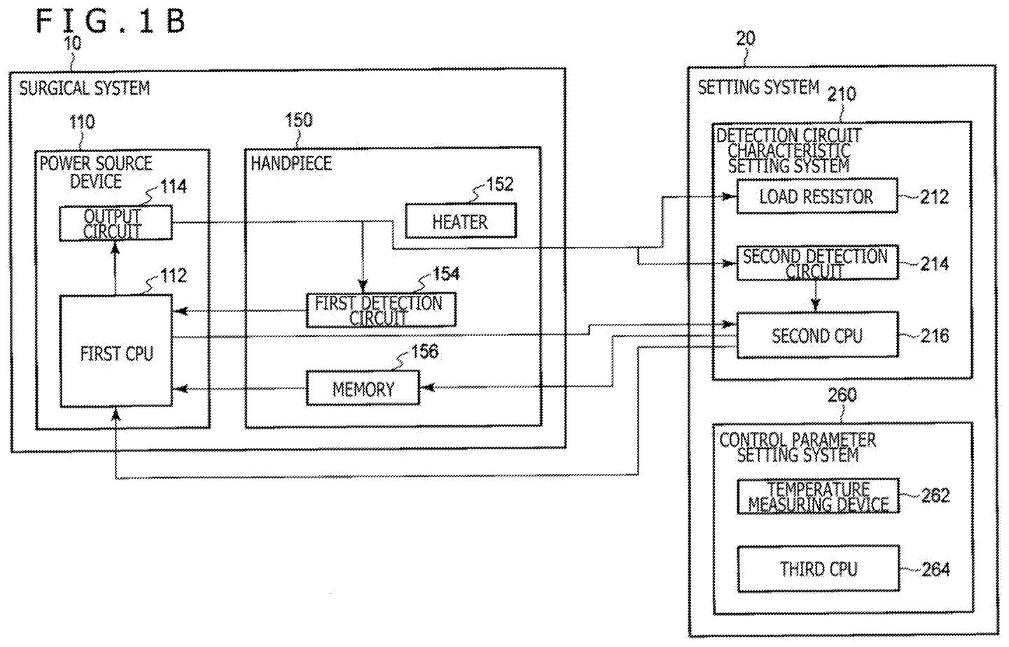

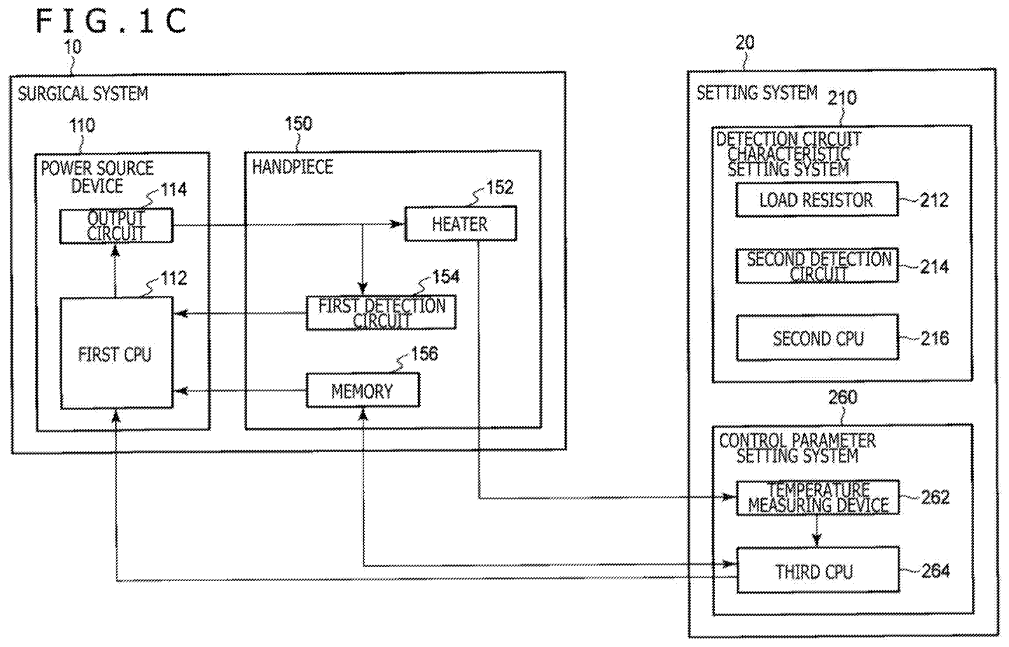

An embodiment of the disclosed technology will be described with reference to the corresponding ones of the drawings. Overviews of configuration examples of a system according to the embodiment are illustrated in FIGS. 1A, 1B and 1C. This embodiment relates to a surgical system 10, and also to a setting system 20 that, for example, is to be used in a manufacturing process of the surgical system 10 and is configured to perform setting of the surgical system 10.

The surgical system 10 is a system for use in treatment of a body tissue, and is used in treatment, for example, to conduct hemostasis or to solidify, seal, cut off or cut open a tissue. The surgical system 10 performs the treatment by applying heat to a body tissue.

The surgical system 10 according to this embodiment includes a handpiece 150 and a power source device 110. The handpiece 150 is used to perform treatment, while the power source device 110 supplies electric power to the handpiece 150. The handpiece 150 is a surgical instrument for surgical treatment, and is configured to penetrate, for example, through the abdominal wall and to perform treatment.

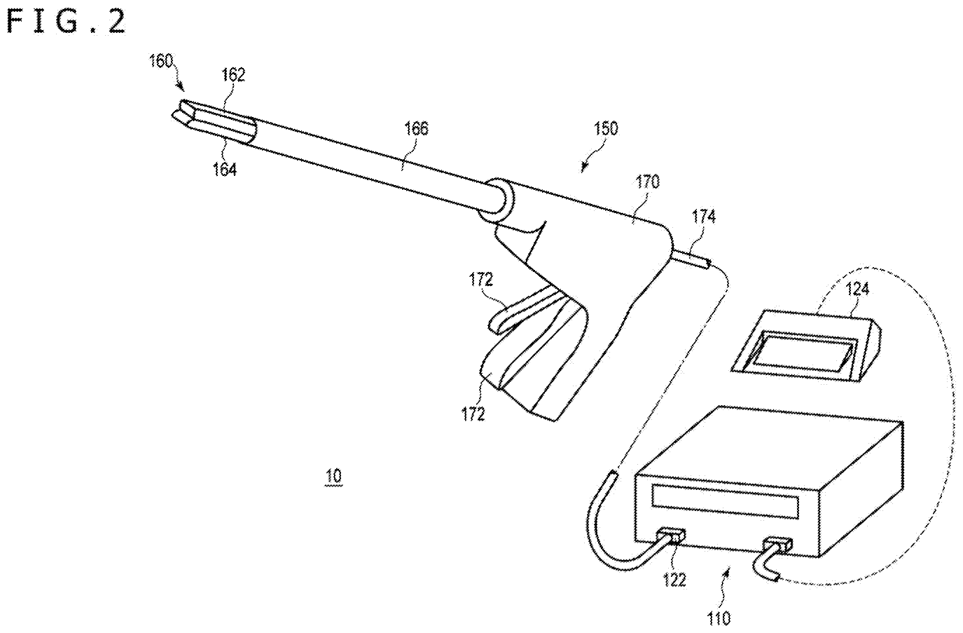

A schematic view of an external appearance of the surgical system 10 according to this embodiment is illustrated in FIG. 2. As illustrated in FIG. 2, the handpiece 150 for the surgical system 10 has a treatment instrument main body 170, a shaft 166 attached to the treatment instrument main body 170, and a grasping portion 160 disposed as an end effector on a distal end of the shaft 166.

The grasping portion 160 has a first grasping member 162 and a second grasping member 164. The first grasping member 162 displaces relative to the second grasping member 164, whereby the grasping portion 160 opens or closes. The grasping portion 160 is configured to grasp a body tissue as a treatment target between the first grasping member 162 and the second grasping member 164. The grasping portion 160 is a portion that grasps the body tissue as the treatment target and applies treatment such as sealing or cut-open of the body tissue.

Both or one of the first grasping member 162 and the second grasping member 164 include or includes a heater 152. When supplied with electric power, the heater 152 generates heat. The heat generated at the heater 152 is transferred to the body tissue, whereby the body tissue is treated.

Operating handles 172 are arranged on the treatment instrument main body 170 to operate the grasping portion 160. The grasping portion 160 is opened or closed, for example, when the operating handles 172 are operated.

The shape of the handpiece 150 as presented herein is an example obviously, and the handpiece 150 may hence have a different shape insofar as it has similar functions. For example, the shape of the treatment instrument main body 170, the length of the shaft 166, the inclusion or non-inclusion of the operating handles 172, the shape of the grasping portion 160, and the like can be changed as needed. Further, the handpiece 150 may be configured, for example, at a distal end portion thereof so that it has no grasping portion, has a trowel shape or the like, and can be pressed against the treatment target. Furthermore, the handpiece 150 can take a variety of forms, for example, a form suited for treatment in the abdominal cavity, a form suited for treatment in a joint cavity, and so on. In addition, the technique of this embodiment can be applied not only to treatment systems for use in rigid endoscopic surgeries as illustrated in FIGS. 1A to 1C but also to treatment systems for use in endoscopic surgeries that employ a flexible endoscope.

The handpiece 150 is connected to the power source device 110 via a cable 174. The power source device 110 supplies electric power to the handpiece 150. This electric power is converted to heat at the heater 152, so that the body tissue grasped by the grasping portion 160 is heated. The cable 174 and the power source device 110 are connected together by a cable connector 122, and this connection is configured to be detachable. Therefore, this surgical system 10 is configured to allow replacement of the handpiece 150 to be used for every treatment. On the other hand, the power source device 110 is arranged at a medical facility, and is used repeatedly.

To the power source device 110, a footswitch 124 is connected. The footswitch 124 which is operated by the foot may be replaced by a handswitch, which is operated by the hand, or other switches. When the footswitch 124 is operated via a pedal thereof by a surgeon, the supply of electric power from the power source device 110 to the handpiece 150 is switched on or off.

As illustrated in FIG. 1A, the power source device 110 includes a first Central Processing Unit (CPU) 112 and a first output circuit 114. The first CPU 112 controls operation of individual portions of the power source device 110. The output circuit 114 adjusts electric power supplied from an unillustrated power source, and then outputs the adjusted electric power to the heater 152 of the handpiece 150 under control of the first CPU 112.

In the surgical system 10 according to this embodiment, a first detection circuit 154, which detects a voltage and a current to be applied to the heater 152, is disposed in the handpiece 150 to control the temperature of the heater 152. The first CPU 112 of the power source device 110 obtains from the first detection circuit 154 values of the voltage and the current to be applied to the heater 152, and by using these values, controls the output of the output circuit 114 under feedback control such as, for example, PID (Proportional-Integral-Differential) control. Described more specifically, the first CPU 112 obtains a resistance value of the heater 152 based on the voltage value and the current values applied to the heater 152. The resistance value of the heater 152 has a correlation with the temperature of the heater 152. Accordingly, the first CPU 112 controls the output of the output circuit 114 so that the resistance value of the heater 152 becomes equal to a resistance value corresponding to a target temperature. Owing to the disposition of the first detection circuit 154 in the handpiece 150 that is close to the heater 152, the voltage and the current to be applied to the heater 152 can be more accurately obtained than its disposition, for example, in the power source device 110.

The handpiece 150 according to this embodiment includes a memory 156. The memory 156 stores values, which are to be used at the first CPU 112 for the control of the surgical system 10, and rely upon individual differences of the handpiece 150. The memory 156 also stores characteristic values for converting, for example, an output value of a sensor, or sensor value, which is included in the first detection circuit 154, to a voltage value or a current value. The memory 156 also stores, for example, control parameters for the control to be performed at the first CPU 112. The control parameters may include, for example, various offset values, values relating to a proportional term, a derivative term or an integral term of PID control, and so on.

In this embodiment, the values which relate to the individual differences of the handpiece 150 and are to be stored in the memory 156 are written, for example, before the shipping of the handpiece 150. For the writing before this shipping, the setting system 20 is used. The setting system 20 includes a detection circuit characteristic setting system 210 and a control parameter setting system 260.

The detection circuit characteristic setting system 210 sets characteristic values for converting each sensor value of the first detection circuit 154 to a voltage value or a current value, in other words, a detection characteristic relating to the first detection circuit 154. The detection characteristic can differ for every first detection circuit 154. The detection circuit characteristic setting system 210 includes a load resistor 212, a second detection circuit 214, and a second CPU 216.

FIG. 1B illustrates a configuration example of a system upon setting the detection characteristic of the first detection circuit 154. The power source device 110 is not the same as the power source device 110 arranged at the medical facility, but has an equivalent function. An output of the output circuit 114 of the power source device 110 is not connected to the heater 152 of the handpiece 150, but is connected to the load resistor 212 of the detection circuit characteristic setting system 210. The second CPU 216 of the detection circuit characteristic setting system 210 instructs the first CPU 112 of the power source device 110 to cause the output circuit 114 to make an output. The load resistor 212 is used as a replacement for the heater 152, and therefore preferably has a resistance value close to that of the heater. Described more specifically, the resistance value of the load resistor 212 may preferably be, for example, about ±10Ω of a maximum resistance value that is expected as the resistance value of the heater 152. As the resistance value of the load resistor 212 is close to the resistance value of the heater 152, it is possible to fully use the output range of the output circuit 114 and the detection range of the first detection circuit 154.

The output from the output circuit 114 to the load resistor 212 is detected by the first detection circuit 154 of the handpiece 150 and the second detection circuit 214 of the detection circuit characteristic setting system 210. The second detection circuit 214 is a calibrated detection circuit, and therefore has a higher detection accuracy than the first detection circuit 154. The detection value of the first detection circuit 154 is transmitted to the first CPU 112. The second CPU 216 obtains the output value of the first detection circuit 154 from the first CPU 112. On the other hand, the second CPU 216 obtains the output value of the second detection circuit 214. The second CPU 216 determines the detection characteristic of the first detection circuit 154 based on the detection results of the first detection circuit 154 and second detection circuit 214. The second CPU 216 stores the determined detection characteristic in the memory 156 of the handpiece 150.

The control parameter setting system 260 sets control parameters relating to the output control of the power source device 110. These control parameters can differ for every handpiece 150. The control parameter setting system 260 includes a temperature measuring device 262 and a third CPU 264.

FIG. 1C illustrates a configuration example of the setting system 20 upon setting a control parameter. In FIG. 1C, the power source device 110 is not the same as the power source device 110 arranged at the medical facility, but also has equivalent functions. The third CPU 264 of the control parameter setting system 260 instructs the first CPU 112 of the power source device 110 to cause the output circuit 114 to make outputs. Here, the third CPU 264 causes the first CPU 112 to perform such test control as maintaining the heater 152 at a predetermined target temperature.

The temperature measuring device 262 of the control parameter setting system 260 obtains the temperature of the heater 152 when electric power is supplied from the output circuit 114 to the heater 152. The temperature measuring device 262 may have a device that obtains the temperature of the heater 152 by contact, for example, like a thermocouple, or may have a device that obtains the temperature of the heater 152 in a contactless manner, for example, like an infrared radiation thermometer. The third CPU 264 obtains information regarding the temperature of the heater 152 from the temperature measuring device 262. Using the information regarding the test control and the information regarding temperature changes obtained at the temperature measuring device 262, the third CPU 264 determines the control parameter to be used at the first CPU 112. The third CPU 264 stores the determined control parameter in the memory 156 of the handpiece 150.

### Setting Procedures

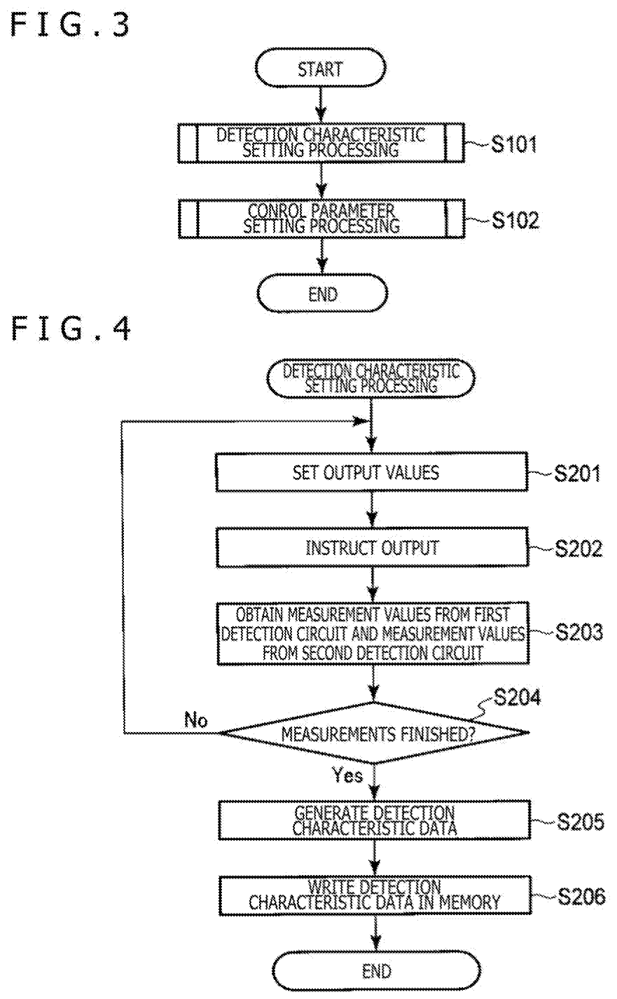

A description will be made about procedures for the setting of a detection characteristic of the first detection circuit 154 of the surgical system 10 according to this embodiment and for the setting of a control parameter for the control to be performed at the first CPU 112. In this embodiment, as illustrated in the flowchart of FIG. 3, detection characteristic setting processing relating to the setting of the detection characteristic of the first detection circuit 154 is first performed in step S101. In the detection characteristic setting processing, the surgical system 10 is connected to the detection circuit characteristic setting system 210 as illustrated in FIG. 1B. Control parameter setting processing relating to the setting of the control parameter at the first CPU 112 is next performed in step S102. In the control parameter setting processing, the surgical system 10 is connected to the control parameter setting system 260 as illustrated in FIG. 1C.

### Detection Characteristic Setting Processing

About the detection characteristic setting processing, a description will be made with reference to the flow chart illustrated in FIG. 4. The processing illustrated in FIG. 4 is performed by the second CPU 216 of the detection circuit characteristic setting system 210.

In step S201, the second CPU 216 sets output values relating to electric power, which is to be outputted from the output circuit 114 of the power source device 110, according to predetermined setting, for example. The output values in this step are successively changed within the ranges of voltages and currents which are expected to be detected at the first detection circuit 154 during use of the surgical system 10.

In step S202, the second CPU 216 sends an output instruction to the first CPU 112 of the power source device 110. Upon reception of the instruction, the first CPU 112 makes the output circuit 114 of the power source device 110 operate so that an output of the intensities set in step S201 is generated from the output circuit 114. At this time, the first detection circuit 154 and the second detection circuit 214 each detect the voltage and the current of electric power to be outputted from the output circuit 114 to the load resistor 212 of the detection circuit characteristic setting system 210 and each generate sensor values representing a voltage value and a current value.

In step S203, the second CPU 216 receives from the first CPU 112 the sensor values that represent the voltage value and the current value generated at the first detection circuit 154. On the other hand, the second CPU 216 obtains the sensor values representing the voltage value and the current value generated at the second detection circuit 214. As the second detection circuit 214 has been calibrated, the second CPU 216 can obtain the output voltage value and the output current value of the output circuit 114 if based on the sensor values of the second detection circuit 214.

In step S204, the second CPU 216 determines whether or not the measurements have been finished. The measurements are determined to have been finished, for example, when the output values set in step S201 have broadly covered the ranges of voltages and currents expected to be detected at the first detection circuit 154 during use of the surgical system 10. If not finished, the processing returns to step S201, where output values are changed and the obtainment of the hereinbefore-mentioned sensor values is repeated.

If the measurements are determined in step S204 to have been finished, the processing proceeds to step S205. In step S205, the second CPU 216 generates detection characteristic data. The detection characteristic data represent correlations between the sensor values of the first detection circuit 154 and the voltage values and the current values, respectively.

In step S206, the second CPU 216 writes the generated detection characteristic data in the memory 156 of the handpiece 150. The detection characteristic setting processing now ends.

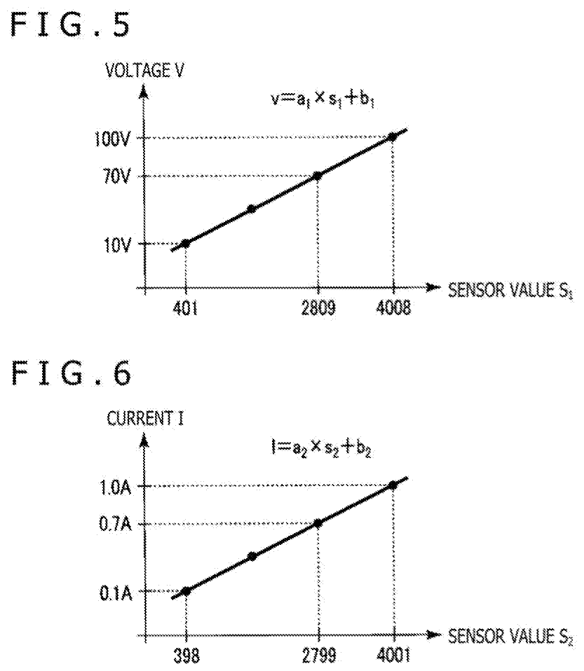

Referring to FIG. 5, a description will be made on the detection characteristic data about the voltage values. In FIG. 5, the abscissa represents the sensor value S₁ corresponding to the voltage value outputted from the first detection circuit 154, while the ordinate represents the voltage value V obtained based on the detection value of the second detection circuit 214. Points indicated by the dots in the graph represent measured values. Based on these points, a correlation has been obtained between the sensor values S₁ of the first detection circuit 154 and the voltage values V. In this example, the voltage values expected to be detected at the first detection circuit 154 during use of the surgical system 10 are assumed to range from 10 to 100 V. In the example of FIG. 5, a correlation of the following linear expression:

V=a1×S1+b1

is obtained using constants a1 and b1. This correlation between the sensor values S₁ of the first detection circuit 154 and the voltage values V is stored as detection characteristic data in the memory 156 of the handpiece 150.

A description on the detection characteristic data about the current values are similar to that of the detection characteristic data about the voltage values. Referring to FIG. 6, a description will be made on the detection characteristic data about the current values. In FIG. 6, the abscissa represents the sensor value S₂ corresponding to the current value outputted from the first detection circuit 154, while the ordinate represents the current value I obtained based on the detection value of the second detection circuit 214. Points indicated by the dots in the graph represent measured values. Based on these points, a correlation has been obtained between the sensor values S₂ of the first detection circuit 154 and the current values I. In this example, the current values expected to be detected at the first detection circuit 154 during use of the surgical system 10 are assumed to range from 0.1 to 1.0 A. In the example of FIG. 6, a correlation of the following linear expression:

I=a2×S2+b2

is obtained using constants a2 and b2. This correlation between the sensor values S₂ of the first detection circuit 154 and the current values I is stored as detection characteristic data in the memory 156 of the handpiece 150.

By using the detection characteristic data that have been stored in the memory 156 as described hereinbefore, the first CPU 112 can accurately determine a voltage value and a current value, which are to be applied to the heater 152, based on sensor values outputted from the first detection circuit 154.

### Control Parameter Setting Processing

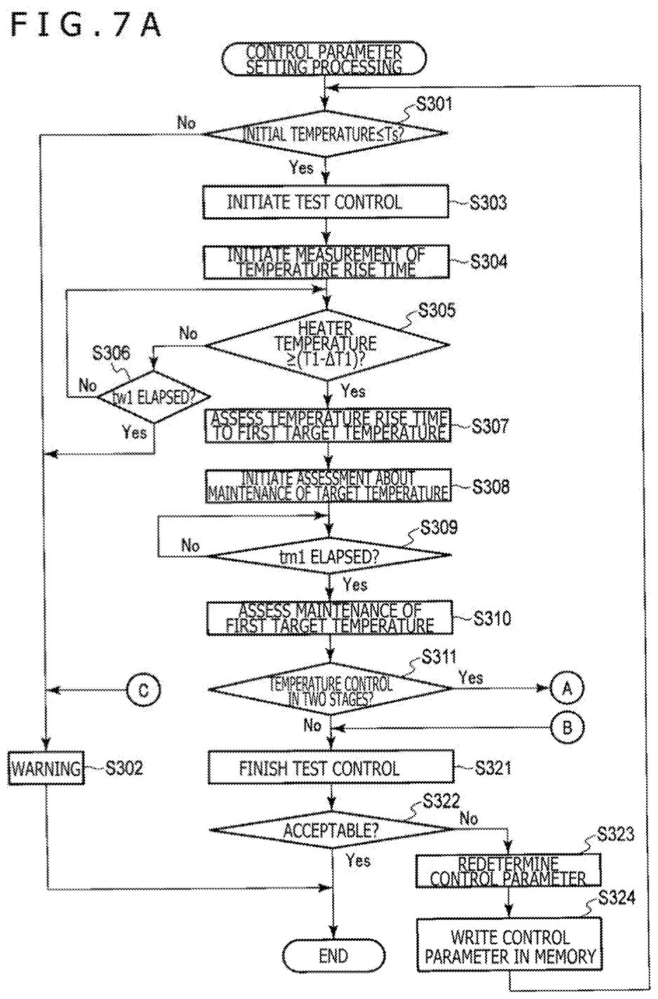

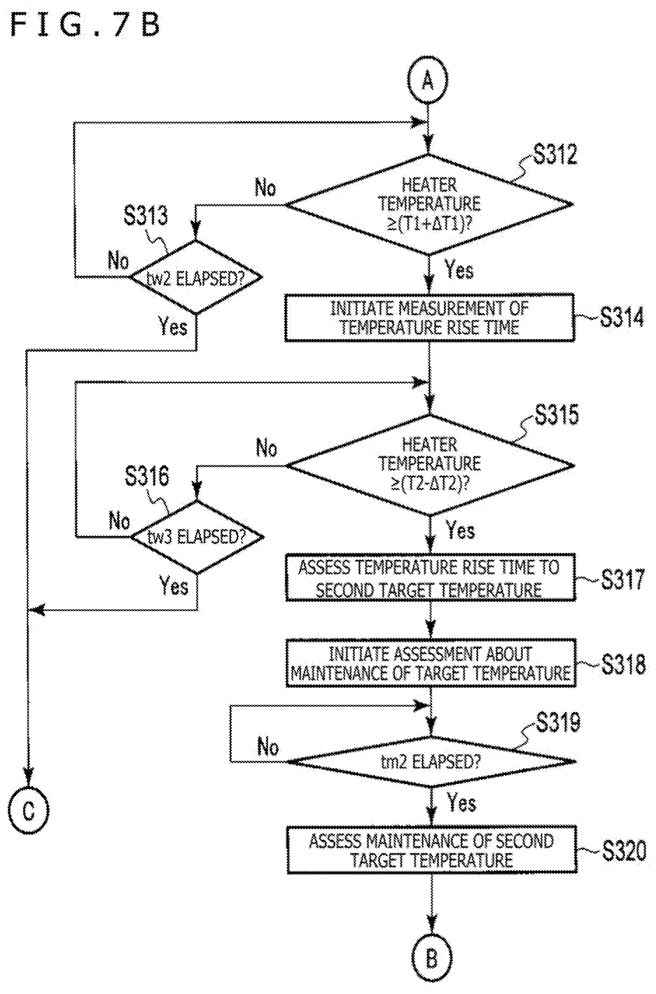

About the control parameter setting processing, a description will be made with reference to the flow chart illustrated in FIGS. 7A and 7B. The processing illustrated in FIGS. 7A and 7B is performed by the third CPU 264 of the control parameter setting system 260. In the control parameter setting processing, the detection characteristics obtained through the detection characteristic setting processing and stored in the memory 156 are used.

In step S301, the third CPU 264 obtains the temperature of the heater 152 from the temperature measuring device 262. Subsequently, the third CPU 264 monitors the temperature of the heater 152. The third CPU 264 determines whether or not the temperature, specifically the initial temperature of the heater 152 is a predetermined temperature Ts or lower. If the initial temperature is not the predetermined temperature Ts or lower, the processing proceeds to step S302. An initial temperature higher than the predetermined temperature Ts involves a potential problem that the setting of the control parameter may not be performed correctly. The control parameter setting processing is therefore suspended if the initial temperature is higher than the predetermined temperature Ts.

Described specifically, the third CPU 264 issues, in step S302, a warning to a user by using unillustrated signaling means. The signaling means may be, for example, a speaker, and may issue a warning sound. As an alternative, the signaling means may be a lamp, which may be turned on as a warning lamp. As a further alternative, the signaling means may be a display, and a warning may be presented on the display. In this further alternative, warning reasons may also be presented along with the warning. For example, it may be presented in step S301 that the initial temperature is higher than the predetermined temperature Ts. After the warning in step S302, the control parameter setting processing ends.

If the initial temperature is determined in step S301 to be the predetermined temperature Ts or lower, the processing proceeds to step S303. In step S303, the third CPU 264 initiates test control of the output from the power source device 110. Described specifically, the third CPU 264 instructs the first CPU 112 so that the output from the output circuit 114 of the power source device 110 is controlled in a predetermined manner. Based on the instruction, the first CPU 112 controls the output of the output circuit 114. In this control, the control parameter stored in the memory 156 are used. In an initial stage, the initial value of the control parameter has been stored in the memory 156.

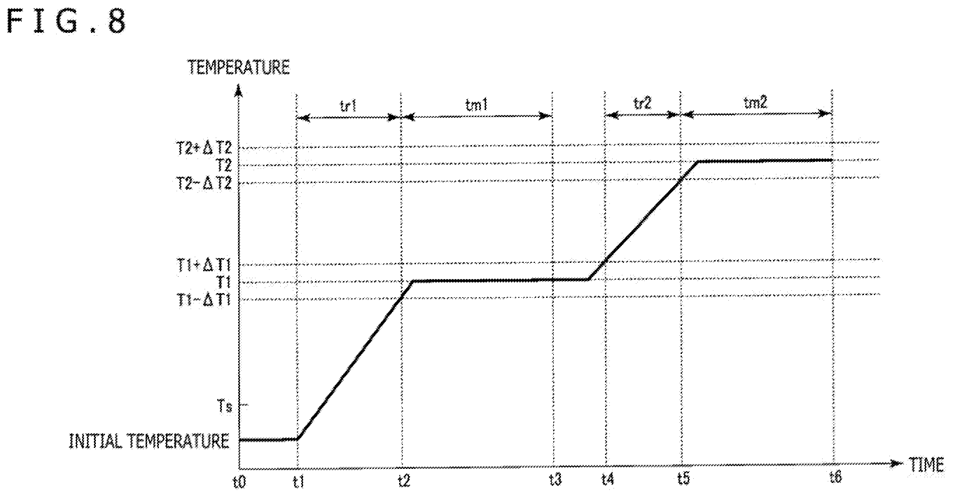

About an example of the test control, a description will be made with reference to a schematic diagram illustrated in FIG. 8. In FIG. 8, the abscissa represents elapsed time, and the ordinate represents the temperature of the heater 152. After the temperature at time t0 which is the time of initiation of the control parameter setting processing, or the initial temperature, has been confirmed to be the predetermined temperature Ts or lower, the application of electric power to the heater 152 is initiated at time t1. Now, the target temperature of the heater 152 is assumed to be a first target temperature T1. By the application of the electric power, the temperature of the heater 152 rises after time t1.

A predetermined temperature difference is assumed to be a first temperature difference ΔT1, and the time when the temperature of the heater 152 has risen to T1−ΔT1, which is lower by the first temperature difference ΔT1 than the first target temperature T1, is assumed to be time t2. The time elapsed from time t1 to time t2 is assumed to be a first temperature rise time tr1. In this embodiment, the length of the first temperature rise time tr1 is assessed.

After time t2, the temperature of the heater 152 is controlled to remain at the first target temperature T1. In this embodiment, it is determined whether or not the temperature of the heater 152 is maintained within a range of T1±ΔT1 around the first target temperature T1 during a first determination period tm1 from time t2 to time t3.

Subsequently, control is performed to raise the target temperature of the heater 152 to a second target temperature T2. At this time, the temperature of the heater 152 rises from the first target temperature T1. The time at which the temperature of the heater 152 has risen to T1+ΔT1 is assumed to be time t4. T1+ΔT1 is higher by the first temperature difference ΔT1 than the first target temperature T1. Assuming that a predetermined temperature difference is a second temperature difference ΔT2, the time at which the temperature of the heater 152 has risen to T2−ΔT is assumed to be time t5. T2−ΔT2 is lower by the second temperature difference ΔT2 than the second target temperature T2. The time elapsed from time t4 to time t5 is assumed to be a second temperature rise time tr2. In this embodiment, the length of the second temperature rise time tr2 is assessed.

After time t5, the temperature of the heater 152 is controlled to remain at the second target temperature T2. In this embodiment, it is determined whether or not the temperature of the heater 152 is maintained within a range of T2±ΔT2 around the second target temperature T2 during a second determination period tm2 from time t5 to time t6. Upon the lapse of the second determination period tm2, the test control finishes.

In the foregoing, the description was made by taking, as an example, the case in which the temperature of the heater 152 is raised to the first target temperature T1, is maintained at the first target temperature T1, is raised to the second target temperature T2, and is maintained at the second target temperature T2. However, this embodiment is not limited to test control such as maintaining the temperature at two stages as described hereinbefore, but single-stage test control may also be performed. The single-stage test control includes only to raise the temperature of the heater 152 to the first target temperature T1, and to maintain it at the first target temperature T1. As the first target temperature T1 to be maintained in the single-stage test control, test control may preferably be performed in two ways, one being a case of the first target temperature T1 of lower temperature in the two-stage temperature control, and the other a case of the second target temperature T2 of higher temperature in the two-stage temperature control.

The surgical system 10 according to this embodiment is configured to enable outputting, for example, two levels of temperatures. Described specifically, the surgical system 10 can perform sealing treatment, that is, can treat a body tissue, which is grasped by the grasping portion 160 of the handpiece 150, at a relatively low temperature to seal the body tissue. Further, the surgical system 10 can also perform cutting treatment, that is, can also treat a body tissue, which is grasped by the grasping portion 160 of the handpiece 150, at a relatively high temperature to cut off the body tissue. The surgical system 10 can perform only sealing treatment, can also perform only cutting treatment, and can also perform sealing treatment and then cutting treatment. Here, the sealing temperature can be, for example, 100° C. or higher but 200° C. or lower. On the other hand, the cutting temperature can be, for example, 200° C. or higher but 300° C. or lower. In the control parameter setting processing, it is possible to perform first control that is single-stage test control in which the first target temperature T1 is set, for example, at 100° C. or higher but 200° C. or lower. In the control parameter setting processing, it is possible to perform second control that is single-stage test control in which the first target temperature T1 is set, for example, at 200° C. or higher but 300° C. or lower. In the control parameter setting processing, it is possible to perform third control that is two-stage test control in which the first target temperature T1 is set, for example, at 100° C. or higher but 200° C. or lower and the second target temperature T2 is set, for example, at 200° C. or higher but 300° C. or lower.

Referring back to FIG. 7A, the description will be continued about the control parameter setting processing. When test control has been initiated in step S303, the time required to raise the temperature of the heater 152 to the target temperature is assessed as mentioned hereinbefore. In step S304, the third CPU 264 initiates measurement of a temperature rise time when the output of the output circuit 114 has entered a phase in which the temperature of the heater 152 is to be raised. The sampling rate for obtaining such temperature data may be set, for example, at ten samples per second or so although not limited thereto.

In step S305, the third CPU 264 determines whether or not the temperature of the heater 152 has risen to T1−ΔT1 or higher. T1−ΔT1 is lower by the first temperature difference ΔT1 than the first target temperature T1. If the temperature of the heater 152 is not T1−ΔT or higher, the processing proceeds to step S306. In step S306, the third CPU 264 determines whether or not the measured temperature rise time has exceeded tw1 as a predetermined time. If the temperature rise time has not exceeded tw1, the processing returns to step S305. If the temperature rise time has exceeded tw1, on the other hand, the processing proceeds to step S302. If the temperature of the heater 152 has not risen to the target temperature despite a lapse of long time, there is a possibility that an abnormality may have occurred somewhere in the surgical system 10 or setting system 20. The control parameter setting processing is therefore suspended if the temperature of the heater 152 has not risen to the target temperature despite the lapse of the long time. Accordingly, the third CPU 264 signals a warning to the user in step S302, and ends the control parameter setting processing. The warning to be signaled to the user may contain information that the temperature of the heater 152 has not risen to the target temperature.

If the temperature of the heater 152 is determined in step S305 to be T1−ΔT1 or higher, the processing proceeds to step S307. In step S307, the third CPU 264 obtains a time at which the temperature of the heater 152 has risen to T1−ΔT1 or higher, and determines the temperature rise time. The third CPU 264 determines whether or not the determined temperature rise time is lower than a predetermined threshold, and if lower than the predetermined threshold, assesses that the handpiece 150 is acceptable in the temperature rise time. If the temperature rise time is equal to or longer than the predetermined threshold, the third CPU 264 assesses that the handpiece 150 is unacceptable in the temperature rise time. Here, the predetermined threshold can be set, for example, at a time in which surgery is smoothly and efficiently performed, for example, at 1 second or so, although not limited thereto.

In step S308, the third CPU 264 initiates control so that the temperature of the heater 152 rises to the first target temperature T1, and initiates an assessment about the maintenance of the target temperature.

In step S309, the third CPU 264 determines whether or not the time elapsed from the initiation of the assessment about the maintenance of the target temperature has exceeded the first determination period tm1. The third CPU 264 repeats the determination of step S309 and remains in a standby state until the first determination period tm1 elapses. When the first determination period tm1 has elapsed, the processing proceeds to step S310.

In step S310, the third CPU 264 makes an assessment about the maintenance of the first target temperature by determining whether or not a predetermined condition has been satisfied in the first determination period tm1. This condition is, for example, a condition for the stability of the temperature of the heater 152. If the temperature of the heater 152 always remains within the range of T1±ΔT1 throughout the first determination period tm1, for example, the third CPU 264 assesses the handpiece 150 as being acceptable in maintaining the temperature of the heater 152 at the first target temperature T1. If the first determination period tm1 includes a time at which the temperature of the heater 152 does not remain within the range of T1±ΔT1, on the other hand, the third CPU 264 assesses the handpiece 150 as being unacceptable in maintaining the heater 152 at the first target temperature T1. Here, ΔT1 may be, for example, 10° C. although not limited thereto.

In step S311, the third CPU 264 determines whether or not the temperature control in the test control is performed in two stages. If the temperature control is performed in two stages, the processing proceeds to step S312. If this is the case, the output of the output circuit 114 is controlled so that the temperature of the heater 152 rises to the second target temperature T2.

In step S312, the third CPU 264 determines whether or not the temperature of the heater 152 has risen to T1±ΔT1 or higher. T1±ΔT1 is higher by the first temperature difference ΔT1 than the first target temperature T1. If the temperature of the heater 152 has not risen to T1±ΔT1 or higher, the processing proceeds to step S313. In step S313, the third CPU 264 determines whether or not the time elapsed from the control of the output of the output circuit 114 to raise the temperature of the heater 152 to the second target temperature T2 has exceeded tw2 as a predetermined time. If the elapsed time has not exceeded tw2, the processing returns to step S312. If the elapsed time has exceeded tw2, on the other hand, the processing proceeds to step S302. If the temperature of the heater 152 does not rise despite a lapse of long time, there is a possibility that an abnormality may have occurred somewhere in the surgical system 10 or setting system 20. The control parameter setting processing is therefore suspended if the temperature of the heater 152 does not rise despite the lapse of the long time. Accordingly, the third CPU 264 signals a warning to the user in step S302, and ends the control parameter setting processing. The warning to be signaled to the user may contain information that the temperature of the heater 152 has not risen to T1±ΔT1.

If the temperature of the heater 152 is determined in step S312 to be T1+ΔT1 or higher, the processing proceeds to step S314. In step S314, the third CPU 264 initiates the measurement of a temperature rise time when the temperature of the heater 152 has risen to T1+ΔT1 or higher.

In step S315, the third CPU 264 determines whether or not the temperature of the heater 152 has risen to T2−ΔT2 or higher. T2−ΔT2 is lower by a second temperature difference ΔT2 than the second target temperature T2. If the temperature of the heater 152 has not risen to T2−ΔT2 or higher, the processing proceeds to step S316. In step S316, the third CPU 264 determines whether or not the measured temperature rise time has exceeded tw3 as a predetermined time. If the temperature rise time has not exceeded tw3, the processing returns to step S315. If the temperature rise time has exceeded tw3, on the other hand, the processing proceeds to step S302. If the temperature of the heater 152 has not risen to the target temperature despite a lapse of long time, there is a possibility that an abnormality may have occurred somewhere in the surgical system 10 or setting system 20. The control parameter setting processing is therefore suspended if the temperature of the heater 152 has not risen to the target temperature despite the lapse of the long time. Accordingly, the third CPU 264 signals a warning to the user in step S302, and ends the control parameter setting processing. The warning to be signaled to the user may contain information that the temperature of the heater 152 has not risen to the target temperature.

If the temperature of the heater 152 is determined in step S315 to be T2−ΔT2 or higher, the processing proceeds to step S317. In step S317, the third CPU 264 obtains a time at which the temperature of the heater 152 has risen to T2−ΔT2 or higher, and determines the temperature rise time. The third CPU 264 determines whether or not the determined temperature rise time is lower than the predetermined threshold, and if lower than the predetermined threshold, assesses that the handpiece 150 is acceptable in the rise time. If the temperature rise time is longer than the predetermined threshold, the third CPU 264 assesses that the handpiece 150 is unacceptable in the temperature rise time.

In step S318, the third CPU 264 initiates control so that the temperature of the heater 152 rises to the second target temperature T2, and initiates an assessment about the maintenance of the target temperature.

In step S319, the third CPU 264 determines whether or not the time elapsed from the initiation of the assessment about the maintenance of the target temperature has exceeded the second determination period tm2. The third CPU 264 repeats the determination of step S319 and remains in a standby state until the second determination period tm2 elapses. When the second determination period tm2 has elapsed, the processing proceeds to step S320.

In step S320, the third CPU 264 makes an assessment about the maintenance of the second target temperature by determining whether or not a predetermined condition has been satisfied in the second determination period tm2. If the temperature of the heater 152 always remains within the range of T2±ΔT2 throughout the second determination period tm2, for example, the third CPU 264 assesses the handpiece 150 as being acceptable in maintaining the temperature of the heater 152 at the second target temperature T2. If the second determination period tm2 includes a time at which the temperature of the heater 152 does not fall within the range of T2±ΔT2, on the other hand, the third CPU 264 assesses the handpiece 150 as being unacceptable in maintaining the heater 152 at the second target temperature T2. Subsequently, the processing proceeds to step S321.

If the temperature control is determined in the determination of step S311 not to have been performed in two stages, the processing proceeds to step S321. In other words, the hereinbefore-mentioned control to raise the temperature of the heater 152 to the second target temperature T2 is not performed.

In step S321, the third CPU 264 instructs the first CPU 112 to finish the test control that controls the temperature of the heater 152. By the finish of the test control, the supply of electric power to the heater 152 is stopped, and the temperature of the heater 152 drops to room temperature or so.

In step S322, the third CPU 264 determines whether or not the handpiece 150 was found to be acceptable in all the assessments made among the assessment about the temperature rise time to the first target temperature T1, the assessment about the operation to maintain at the first target temperature T1, the assessment about the temperature rise time to the second target temperature T2, and the assessment about the operation to maintain at the second target temperature T2, all of which have been mentioned hereinbefore. If it is determined that the handpiece 150 was found to be acceptable in all the assessments made, the control parameter setting processing ends.

If it is determined through the determination of step S322 that the handpiece 150 was found to be unacceptable in at least one assessment, the processing proceeds to step S323. In step S323, the third CPU 264 redetermines the control parameter according to a predetermined rule. In step S324, the third CPU 264 writes the redetermined control parameter in the memory 156, and the processing returns to step S301. Accordingly, the hereinbefore-mentioned tests and assessments are performed using the redetermined control parameter. In this manner, the control parameter is adjusted until the handpiece 150 is found to be acceptable in all the assessments.

The control parameter setting processing as described hereinbefore performs at least one of two-stage test control using a first target temperature as a sealing temperature and a second target temperature as a cutting temperature, single-stage test control using the first target temperature as the sealing temperature, and single-stage test control using the first target temperature as the cutting temperature. For example, control is performed three times, including the two-stage test control using the first target temperature as the sealing temperature and the second target temperature as the cutting temperature, the single-stage test control using the first target temperature as the sealing temperature, and the single-stage test control using the first target temperature as the cutting temperature. Based on the results of the respective tests, the control parameters are determined, and the individual control parameters are stored in the memory 156. By providing these three kinds of control parameters, each control is improved in accuracy. It is hence preferred to perform the three kinds of tests.

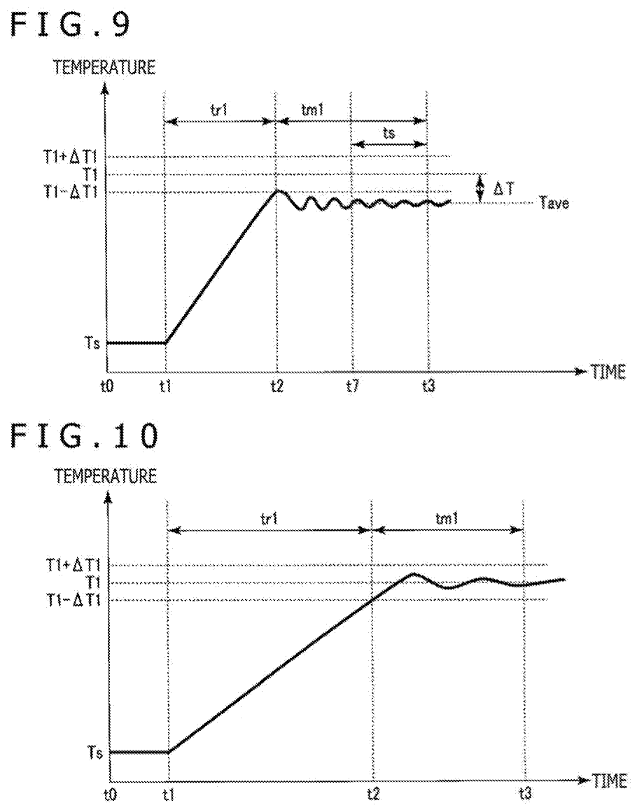

With reference to FIGS. 9 to 13, a description will be made about various examples of a redermination method of the control parameter. The examples to be described hereinafter are illustrative, and other methods may be used for the redetermination of the control parameter. In each of FIGS. 9 to 13, the abscissa represents the elapsed time, and the ordinate represents the temperature of the heater 152.

As illustrated in FIG. 9, for example, control is made so that from time t1, the temperature of the heater 152 rises towards the first target temperature T1. The temperature of the heater 152 is now assumed to have reached T1−ΔT1 at time t2. Under the test control, the temperature of the heater 152 is subsequently controlled to remain at the first target temperature T1. In the example of FIG. 9, however, the average of the actual temperature of the heater 152 is Tave. Comparing Tave, the average of the actual temperature of the heater 152, with the first target temperature T1, there is only a difference of ΔT. If there is such a temperature difference ΔT, an offset is added to the control value to eliminate the temperature difference ΔT. Described more specifically, the temperature of the heater 152, for example, is controlled by measuring the resistance value of the heater 152, the resistance value being proportional to the temperature, and using the resistance value. Therefore, an offset resistance value that is equivalent to the temperature difference is added to a target resistance value corresponding to the first target temperature T1. The calculation of Tave may be made over the entirety of the first determination period tm1. Preferably, however, the calculation of Tave may made using data of a stable period ts ranging from time t7 to time t3 as a latter half of the first determination period tm1. In the stable period ts, the temperature is more stable and is insusceptible, for example, to an inappropriate PID parameter.

FIG. 10 illustrates a case in which the temperature rise rate was low and the first temperature rise time tr1 was longer than a predetermined period. The first temperature rise time tr1 is a period ranging from time t1, at which the temperature of the heater 152 was begun to rise, to time t2, at which the temperature of the heater 152 rose to T1−ΔT1 or higher. If the temperature rise time is longer than the predetermined period as illustrated in FIG. 10 and the handpiece 150 is assessed to be unacceptable, a parameter adjustment is made to increase the proportional term of the PID control parameter or to decrease the derivative term of the PID control parameter. The increment or the decrement of each term may be a preset fixed value, a difference between the predetermined period and the temperature rise time, in other words, a value proportional to a delay of the temperature rise time from the predetermined period, or another value.

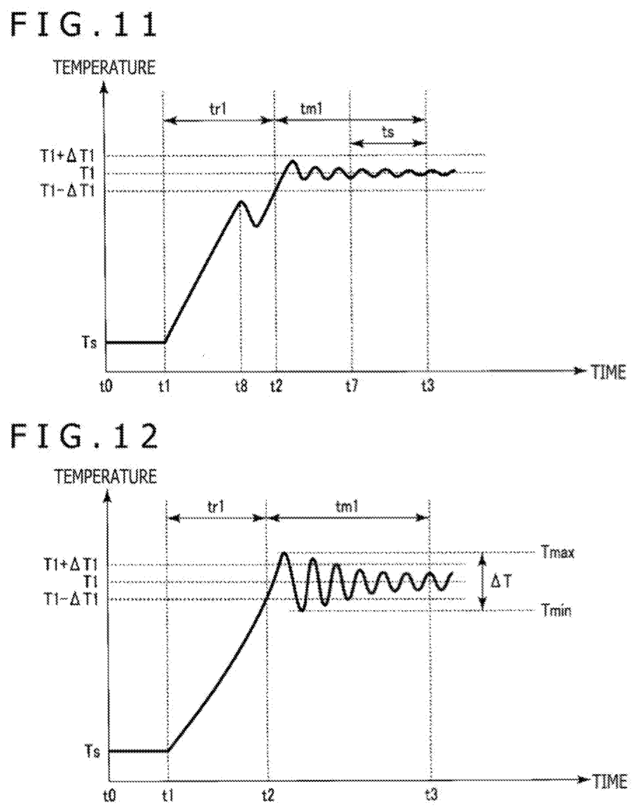

FIG. 11 illustrates a case in which suppression of temperature rise was strong upon raising the temperature and the temperature of the heater 152 took a local maximum value at time t8 when the temperature of the heater 152 arose close to T1−ΔT1, the temperature of the heater 152 then once dropped, and subsequently the temperature of the heater 152 again increased to rise to T1−ΔT1 or higher at time t2. If, as illustrated in FIG. 11, for example, the suppression of the temperature rise was too strong upon raising the temperature, the temperature rise time was longer than the predetermined period and the handpiece 150 was assessed to be unacceptable, a parameter adjustment is made to increase the proportional term of the PID control parameter or to decrease the derivative term of the PID control parameter as in the case illustrated in FIG. 10. The increment or the decrement of each term may be a preset fixed value, or another value. Further, the assessment of the temperature rise time may also be configured so that, in such a case in which the temperature of the heater 152 takes a local maximum value during raising the temperature, as illustrated in FIG. 11, the handpiece 150 is assessed to be unacceptable irrespective of the temperature rise time.

FIG. 12 illustrates a case in which, in the first determination period tm1 where the temperature of the heater 152 was controlled to remain at the first target temperature T1, the temperature of the heater 152 varied beyond the permissible range of T1±ΔT1 and the difference ΔT between a maximum value and a minimum value of the temperature became greater than 2×ΔT1. If the handpiece 150 is found to be unacceptable in the assessment about the maintenance of the target temperature as illustrated in FIG. 12, a parameter adjustment is made to increase the derivative term of the PID control parameter or to decrease the proportional term of the PID control parameter. The increment or the decrement of each term may be a preset fixed value, a value corresponding to the instability of the control such as a value proportional to the difference between ΔT, the difference between a maximum value and a minimum value among measured temperatures, and 2×ΔT1, the width of permissible temperatures, or another value.

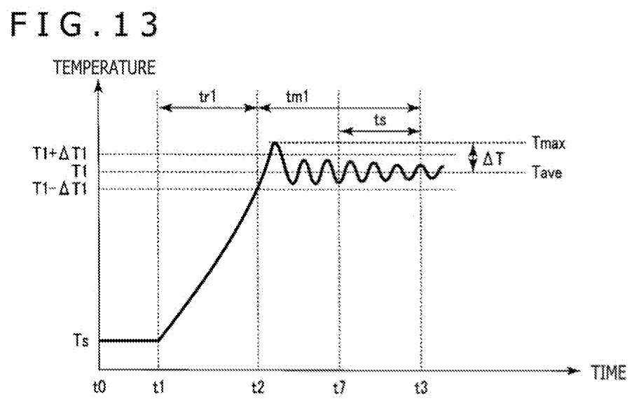

FIG. 13 illustrates a case in which an excessively large overshoot occurred after the temperature of the heater 152 had arisen to T1−ΔT1 or higher. In such a case, the difference ΔT between the maximum value Tmax in the first determination period tm1 for the temperature of the heater 152 and the average value Tave over the stable period is becomes greater than a predetermined value. If an overshoot is excessively large as illustrated in FIG. 13, a parameter adjustment is made to increase the derivative term of the PID control parameter or to decrease the proportional term of the PID control parameter as in the case illustrated in FIG. 12. The increment or the decrement of each term may be a preset fixed value, a value corresponding to the magnitude of the overshoot such as a value proportional to the difference ΔT between the maximum value Tmax and the average value Tave among the measured temperatures, or another value.

As described hereinbefore, appropriate detection characteristic data for the first detection circuit 154 are stored in the memory 156 through the detection characteristic setting processing, and a suitable control parameter for the feedback control to be performed at the first CPU 112 is stored in the memory 156 through the control parameter setting processing. Using the data stored in the memory 156, high-accuracy control can be realized by the power source device 110 with respect to the temperature of the heater 152 of the handpiece 150.

### Operation of Surgical System

A description will be made about operation when the surgical system 10 according to this embodiment is used a surgery. A surgeon uses the surgical system 10 by connecting the obtained handpiece 150 to the power source device 110 arranged at the medical facility. The surgeon first operates an unillustrated input device of the power source device 110 to set output conditions for the surgical system 10, such as output target values, treatment time and the like relating to treatment. The output conditions may be configured to separately set values for every parameter, or to select a set of setting values according to a surgical method.

The grasping portion 160 and shaft 166 of the handpiece 150 are inserted, for example, into an abdominal cavity through the abdominal wall. The surgeon controls the operating handles 172 to open and close the grasping portion 160 so that a body tissue is grasped as a treatment target between the first grasping member 162 and the second grasping member 164.

After grasping the body tissue as the treatment target by the grasping portion 160, the surgeon operates the footswitch 124. When the footswitch 124 is switched on, electric power is supplied from the power source device 110 to the heater 152 of the handpiece 150 via the cable 174. Using this electric power, the handpiece 150 applies thermal energy to the body tissue as the treatment target, and treats the body tissue.

At this time, the first CPU 112 of the power source device 110 monitors the value of electric power, which is to be supplied to the heater 152, by using the first detection circuit 154. Here, the detection characteristics stored in the memory 156 are used. The first CPU 112 performs feedback control on the output of the output circuit 114. For example, the first CPU 112 determines a voltage value and a current value to be applied to the heater 152, and from these values, calculates the resistance value of the heater 152. This resistance value represents the temperature of the heater 152. The first CPU 112 performs output control of the output circuit 114 so that the temperature of the heater 152 remains at the target temperature. In this output control, the first CPU 112 uses the control parameters stored in the memory 156. When desired energy is applied to the body tissue and the treatment of the body tissue finishes, the supply of electric power to the handpiece 150 is stopped. By the foregoing, the treatment of the body tissue is completed.

### Advantages of System

In the surgical system 10 according to this embodiment, handpieces 150 are used with replacement for every surgery in combination with the same single power source device 110. Each handpiece 150 has various individual differences including those in the characteristics of the first detection circuit 154. According to this embodiment, detection characteristic data and control parameters, which correspond to such individual differences, are stored in the memory 156 disposed in the handpiece 150. The first CPU 112 of the power source device 110, which controls the output of the handpiece 150, hence performs the control of the output by reading the values stored in the memory 156. Consequently, the operation control of each handpiece 150 can be precisely performed despite the existence of individual difference in each handpiece 150.

Further, with the use of the setting system 20, the detection characteristic setting processing and control parameter setting processing can be automatically performed in short time according to the predetermined processing procedures. The productivity of the handpiece 150 is therefore not impaired.

### First Modification

A description will be made about a first modification of the hereinbefore-mentioned embodiment. Here, the description is made about differences from the hereinbefore-mentioned embodiment, and the same elements are identified by the same numeral references, and their description is omitted. In the hereinbefore-mentioned embodiment, the power source device 110 that supplies electric power to the handpiece 150 in the detection characteristic setting processing and the control parameter setting processing is the power source device 110 used in the surgical system 10. Different from the hereinbefore-mentioned embodiment, the power source device for use in the detection characteristic setting processing and the control parameter setting processing is not required to be the power source device used in the surgical system 10, but may be any power source device insofar as its output is adjustable.

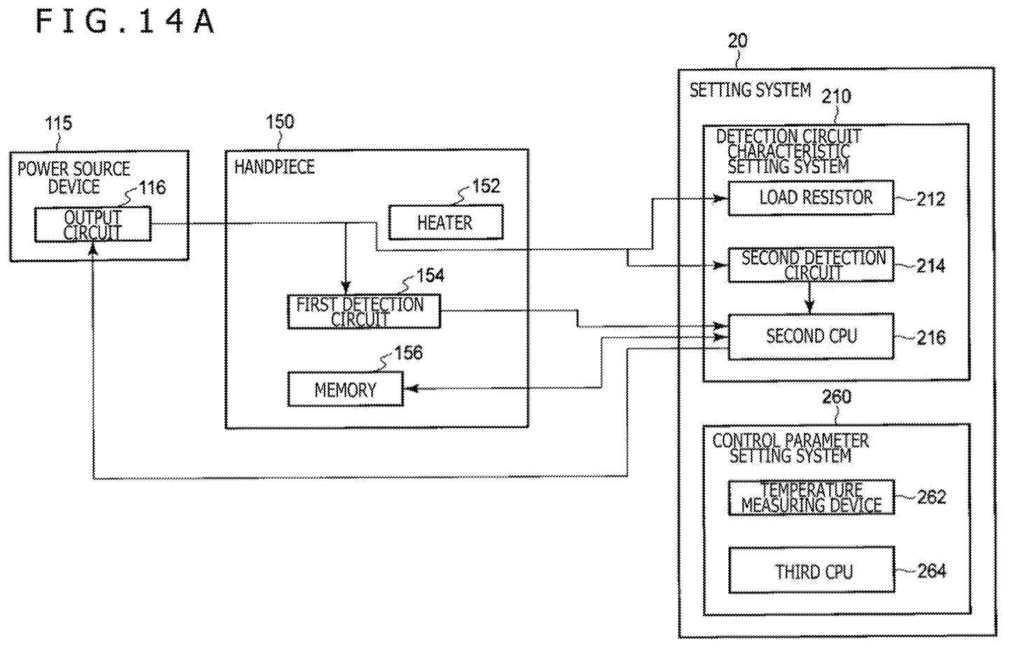

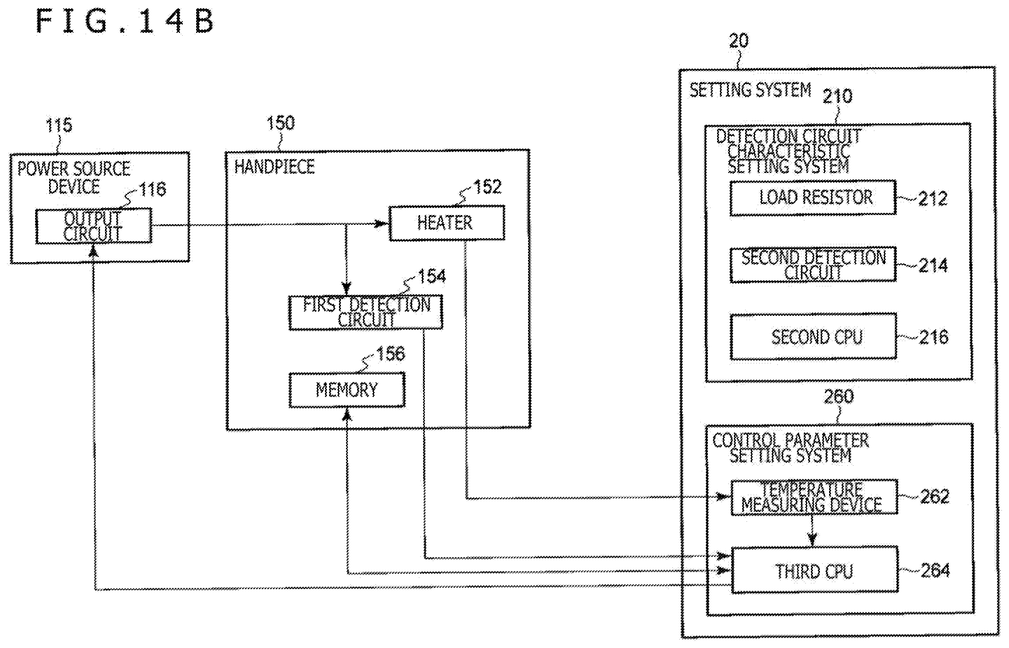

FIGS. 14A and 14B each illustrate an overview of configuration examples of this modification. In this modification, a power source device 115 for use in the detection characteristic setting processing and the control parameter setting processing has a general output circuit 116, the output of which is adjustable. In contrast, the power source device 115 in this modification does not have the first CPU 112 configured to appropriately control the temperature of the heater 152 as in the power source device 110 of the surgical system 10.

In the detection characteristic setting processing in this modification as illustrated in FIG. 14A, the second CPU 216 of the detection circuit characteristic setting system 210 reads various data stored in the memory 156 of the handpiece 150, and also obtains sensor values outputted from the first detection circuit 154. Using such information, the second CPU 216 controls the output of the output circuit 116 of the power source device 115. Detection characteristic data obtained through the detection characteristic setting processing controlled as mentioned hereinbefore is written in the memory 156.

In the control parameter setting processing in this modification as illustrated in FIG. 14B, the third CPU 264 of the control parameter setting system 260 reads various data stored in the memory 156 of the handpiece 150, and also obtains sensor values outputted from the first detection circuit 154. Using such information, the third CPU 264 controls the output of the output circuit 116 of the power source device 115. The control parameters obtained through the control parameter setting processing controlled as described hereinbefore is written in the memory 156.

According to this modification, similar setting is also performed as in the hereinbefore-mentioned embodiment, and similar advantageous effects are also available as in the hereinbefore-mentioned embodiment.

### Second Modification

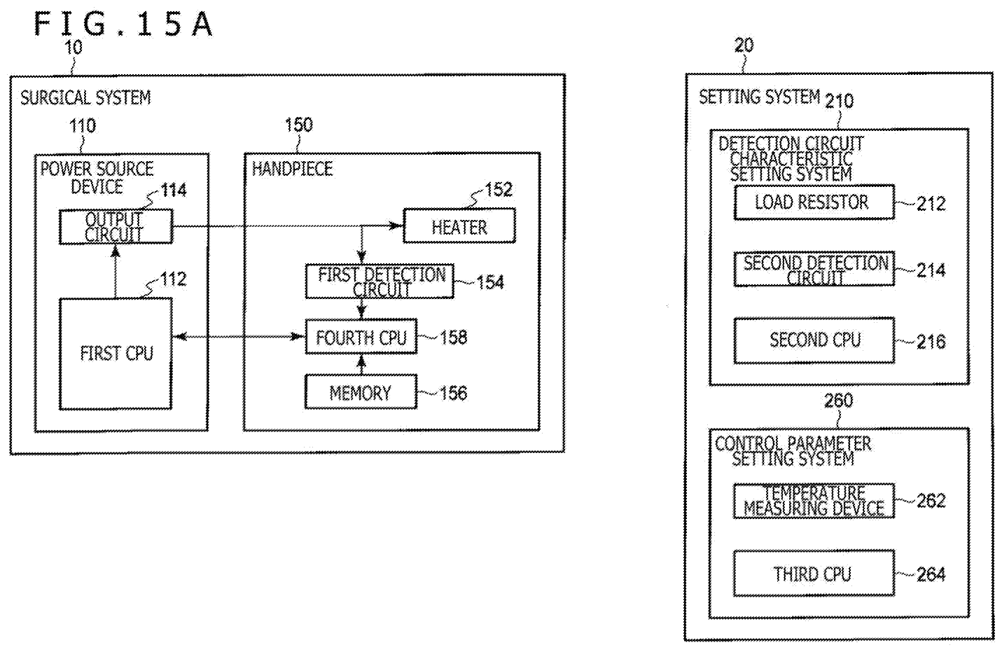

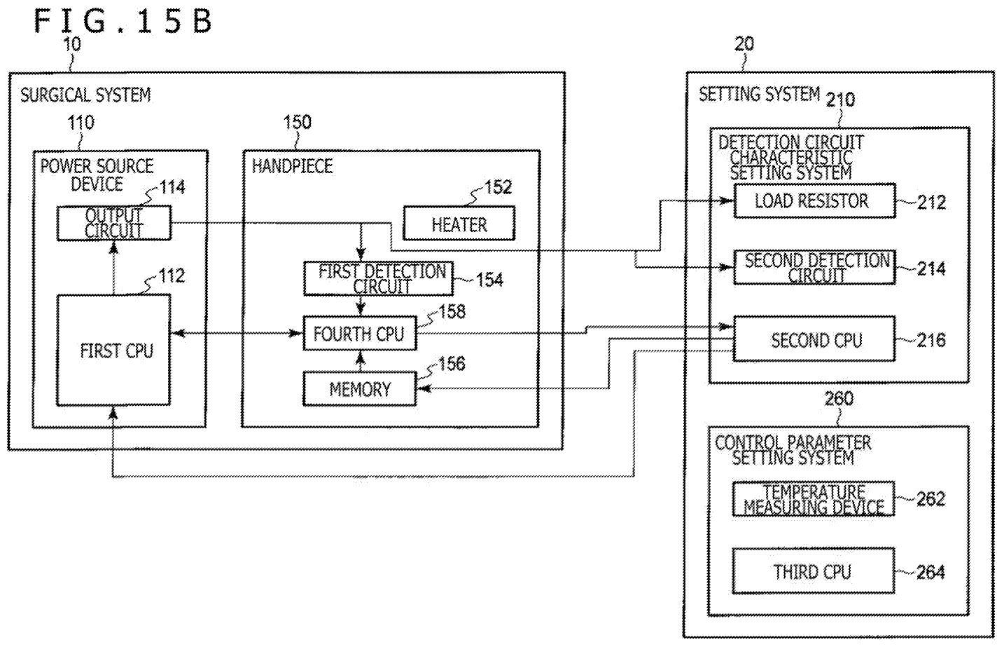

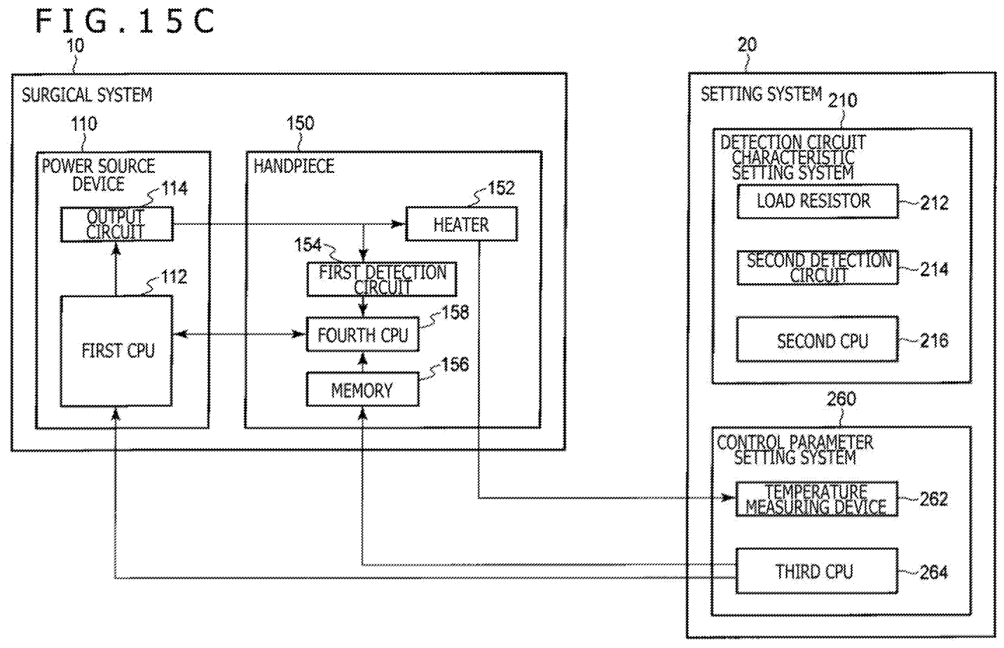

A description will be made about a second modification of the hereinbefore-mentioned embodiment. Here, the description is made about differences from the hereinbefore-mentioned embodiment, and the same elements are identified by the same numeral references, and their description is omitted. In the hereinbefore-mentioned embodiment, the control of the output of the power source device 110 is performed at the first CPU 112 disposed in the power source device 110. However, this modification is configured so that as illustrated in FIG. 15A, the control of the output of the power source device 110 is partly performed at a fourth CPU 158 disposed in the handpiece 150. The fourth CPU 158 reads the detection characteristic data, control parameter and the like from the memory 156, and performs output control by using such information, sensor values outputted from the first detection circuit 154, and also an instruction for the initiation or stop of an output or a like instruction as obtained from the first CPU 112. The fourth CPU 158 outputs, to the first CPU 112, an instruction for the output from the output circuit 114 of the power source device 110, and based on the instruction, the first CPU 112 causes the output circuit 114 to operate.

In such detection characteristic setting processing at the surgical system 10, the second CPU 216 of the detection circuit characteristic setting system 210 obtains the information, which was obtained from the first CPU 112 of the power source device 110 in the hereinbefore-mentioned embodiment, from the fourth CPU 158 of the handpiece 150 as illustrated in FIG. 15B. Other operations are similar to those in the hereinbefore-mentioned embodiment.

A system configuration in the control parameter setting processing at the surgical system 10 according to this modification is illustrated in FIG. 15C. The third CPU 264 of the control parameter setting system 260 determines the control parameter for use at the first CPU 112 of the power source device 110 in the hereinbefore-mentioned embodiment, but determines the control parameter for use at the fourth CPU 158 of the handpiece 150 in this modification. Other operations are similar to those in the hereinbefore-mentioned embodiment.

According to this modification, similar setting is also performed as in the hereinbefore-mentioned embodiment, and similar advantageous effects are also available as in the hereinbefore-mentioned embodiment.

### Other Modifications

Instead of the CPUs such as the first CPU 112, the second CPU 216, the third CPU 264 and the fourth CPU 158, a dedicated integrated circuit, for example, such as an Application Specific Integrated Circuit (ASIC) or Field Programmable Gate Array (FPGA) may be used.

Further, the functions of the second CPU 216 of detection circuit characteristic setting system 210 and the functions of the third CPU 264 of the control parameter setting system 260 may be served, for example, by a single CPU or the like.

In the hereinbefore-mentioned embodiment, the example is presented in which the control parameter setting processing is performed after the detection characteristic setting processing. However, the order of these processes may be reversed. In the control parameter setting processing, tentative values may be used as the detection characteristic data of the first detection circuit 154. It is, however, preferred to also use the detection characteristic data, which have been determined in the detection characteristic setting processing, in the control parameter setting processing, because higher accuracy is available in the control. In addition, only one of the detection characteristic setting processing and the control parameter setting processing may be performed.

In the hereinbefore-mentioned embodiment, the example is presented in which the detection characteristic setting processing and the control parameter setting processing are performed before the shipping of the handpiece 150, for example, at a factory. In other word, the case in which the detection characteristic setting processing and the control parameter setting processing are part of the manufacturing processes of the handpiece 150 was presented as an example. The disclosed technology is, however, not limited to such an example or a case. The detection characteristic setting processing and the control parameter setting processing may be performed as adjustments of the handpiece 150 at a medical facility before the handpiece 150 is used. In such a case, the functions of the setting system 20 may be implemented in the power source device 110 arranged, for example, at a medical facility.

In the hereinbefore-mentioned embodiment, the example is presented in which the handpiece 150 includes the heater 152 and the body tissue grasped by the grasping portion 160 is treated with heat generated by the heater 152. However, the energy to be applied from the handpiece 150 to the body tissue as a treatment target is not limited to such heat. For example, the handpiece may be a high-frequency treatment instrument such as that to be described next. Described specifically, the first grasping member 162 and the second grasping member 164 include electrodes on their surfaces to be brought into contact with a body tissue, and a high-frequency voltage is applied between the electrodes. A current flows through the body tissue grasped between the electrodes, whereby the body tissue is heated and treated. As an alternative, the handpiece may be an ultrasonic treatment instrument such as that to be described next. Described specifically, the second grasping member 164 is connected to an ultrasonic transducer, and the second grasping member 164 which is brought into contact with a body tissue is ultrasonically vibrated. Under friction between the second grasping member and the body tissue, the body tissue is treated. As a further alternative, the handpiece may be such one that a body tissue is treated with two or more kinds of energy among heat, high-frequency electric power and ultrasonic vibrations. These handpieces each have a similar configuration as one of the hereinbefore-mentioned embodiment and the modifications thereof. Characteristics corresponding to an output are obtained and stored in a memory, whereby similar advantageous effects are obtained.

In sum, the disclosed technology is directed to a method of determining setting parameters for temperature control of a handpiece used in a surgical system. The handpiece includes a heater, a first detection circuit configured to measure a voltage and a current to be supplied to the heater, and a memory. The method comprises obtaining a detection characteristic representing a correlation between a sensor value of the first detection circuit and the at least one of the voltage value and the current value. The correlation is used for determination of the at least one of the voltage value and the current value being applied to the heater and based on a result measured using a second detection circuit having a higher detection accuracy than the first detection; storing the detection characteristic in the memory; obtaining temperature changes of the heater while performing a test control that supplies electric power to the heater to control a temperature of the heater in a predetermined manner; determining, based on the temperature changes, a control parameter which is to be used to control the temperature of the heater; and storing the control parameter in the memory.

The control parameter is determined after obtaining the detection characteristics and the temperature changes of the heater are obtained using the detection characteristics. The detection characteristics of the first detection circuit are obtained by measuring with each of the first detection circuit and the second detection circuit, a voltage and a current which are to be supplied to a load resistor having a resistance value of a maximum resistance value of the heater ±10Ω. The control parameter is determined by repeating changing the control parameter according to a predetermined rule until the temperature changes of the heater satisfy a predetermined determination condition while the test control is performed if the temperature changes of the heater do not satisfy the predetermined determination condition while the test control is performed. The control parameter includes an offset value or a proportional term, a derivative term, or an integral term of proportional-integral-differential control. The determination condition includes at least one of a condition about a duration time of a temperature rise that is a time required to raise the temperature of the heater to a predetermined value and a condition about stability of the temperature of the heater when the test control is performed to maintain the temperature of the heater at a predetermined target temperature.

The condition about the duration time of the temperature rise is one second or shorter and the condition about the stability is that the temperature of the heater has a highest temperature and a lowest temperature between which a difference of 10° C. or less exists, when the test control is performed so that the temperature of the heater is maintained at the predetermined target temperature. The test control includes a first control of raising the temperature of the heater to a sealing temperature and maintaining the heater at the sealing temperature. A second control of raising the temperature of the heater to a cutting temperature and maintaining the heater at the cutting temperature. A third control of raising the temperature of the heater to the sealing temperature and maintaining the heater at the sealing temperature and then raising the temperature of the heater to the cutting temperature and maintaining the heater at the cutting temperature. The control parameter includes a first parameter for the first control, a second parameter for the second control, and a third parameter for the third control. The sealing temperature is 100° C. or higher but 200° C. or lower and the cutting temperature is 200° C. or higher but 300° C. or lower.

Another aspect of the disclosed technology is directed to a handpiece for a surgical system. The handpiece comprises a heater; a first detection circuit configured to measure a voltage and a current to be supplied to the heater; and a memory. The memory stores detection characteristics representing a correlation between a sensor value of the first detection circuit and the at least one of the voltage value and the current value. The correlation being used for determination of the at least one of the voltage value and the current value being applied to the heater as obtained based on results measured using a second detection circuit having a higher detection accuracy than the first detection circuit. A control parameter which is to be used to control a temperature of the heater and is determined based on temperature changes of the heater while performing a test control that supplies electric power to the heater to control the temperature of the heater in a predetermined manner.

The control parameter is determined after obtaining the detection characteristics, and the temperature changes of the heater are obtained using the detection characteristics. The detection characteristics of the first detection circuit are obtained by measuring with each of the first detection circuit and the second detection circuit, a voltage and a current which are to be supplied to a load resistor having a resistance value of a maximum resistance value of the heater ±10Ω. The control parameter is determined by repeating changing the control parameter according to a predetermined rule until the temperature changes of the heater satisfy a predetermined determination condition while the test control is performed if the temperature changes of the heater do not satisfy the predetermined determination condition while the test control is performed. The control parameter includes an offset value or a proportional term, a derivative term, and/or an integral term of proportional-integral-differential control. The determination condition includes at least one of a condition about a duration time of a temperature rise that is a time required to raise the temperature of the heater to a predetermined value and a condition about stability of the temperature of the heater when the test control is performed to maintain the temperature of the heater at a predetermined target temperature.

The condition about the duration time of the temperature rise is one second or shorter and the condition about the stability is that the temperature of the heater has a highest temperature and a lowest temperature, between which a difference of 10° C. or less exists, when the test control is performed so that the temperature of the heater is maintained at the predetermined target temperature. The test control includes a first control of raising the temperature of the heater to a sealing temperature and maintaining the heater at the sealing temperature. A second control of raising the temperature of the heater to a cutting temperature and maintaining the heater at the cutting temperature. A third control of raising the temperature of the heater to the sealing temperature and maintaining the heater at the sealing temperature and then raising the temperature of the heater to the cutting temperature and maintaining the heater at the cutting temperature. The control parameter includes a first parameter for the first control, a second parameter for the second control, and a third parameter for the third control. The sealing temperature is 100° C. or higher but 200° C. or lower, and the cutting temperature is 200° C. or higher but 300° C. or lower.

A further aspect of the disclosed technology is directed to a surgical system comprises a handpiece having a heater; a first detection circuit configured to measure a voltage and a current to be supplied to the heater, and a memory. The memory stores detection characteristics representing a correlation between a sensor value of the first detection circuit and the at least one of the voltage value and the current value. The correlation is used for determination of the at least one of the voltage value and the current value being applied to the heater as obtained based on results measured using a second detection circuit having a higher detection accuracy than the first detection circuit. A control parameter which is to be used to control a temperature of the heater and is determined based on temperature changes of the heater while performing a test control that supplies electric power to the heater to control the temperature of the heater in a predetermined manner. A power source device configured to supply electric power to the heater of the handpiece. The power source device is configured to obtain the detection characteristics and the control parameter from the memory of the handpiece and control the electric power which is to be supplied to the heater, based on a sensor value obtained by the first detection circuit, the detection characteristics, and the control parameter.

While various embodiments of the disclosed technology have been described above, it should be understood that they have been presented by way of example only, and not of limitation. Likewise, the various diagrams may depict an example schematic or other configuration for the disclosed technology, which is done to aid in understanding the features and functionality that can be included in the disclosed technology. The disclosed technology is not restricted to the illustrated example schematic or configurations, but the desired features can be implemented using a variety of alternative illustrations and configurations. Indeed, it will be apparent to one of skill in the art how alternative functional, logical or physical locations and configurations can be implemented to implement the desired features of the technology disclosed herein.

Although the disclosed technology is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the disclosed technology, whether or not such embodiments are described and whether or not such features are presented as being a part of a described embodiment. Thus, the breadth and scope of the technology disclosed herein should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent.

Additionally, the various embodiments set forth herein are described in terms of exemplary schematics, block diagrams, and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular configuration.

What is claimed is:

1. A method of determining setting parameters for temperature control of a handpiece used in a surgical system, the handpiece including a heater, a first detection circuit configured to measure a voltage and a current to be supplied to the heater, and a memory, the method comprising:
obtaining a detection characteristic representing a correlation between a sensor value of the first detection circuit and the at least one of the voltage value and the current value, the correlation being used for determination of the at least one of the voltage value and the current value being applied to the heater and based on a result measured using a second detection circuit having a higher detection accuracy than the first detection;
storing the detection characteristic in the memory;
obtaining temperature changes of the heater while performing a test control that supplies electric power to the heater to control a temperature of the heater in a predetermined manner;
determining, based on the temperature changes, a control parameter which is to be used to control the temperature of the heater; and
storing the control parameter in the memory.

2. The method of claim 1, wherein the control parameter is determined after obtaining the detection characteristics, and the temperature changes of the heater are obtained using the detection characteristics.

3. The setting method of claim 1, wherein the detection characteristics of the first detection circuit are obtained by measuring, with each of the first detection circuit and the second detection circuit, a voltage and a current which are to be supplied to a load resistor having a resistance value of a maximum resistance value of the heater ±10Ω.

4. The method of claim 1, wherein the control parameter is determined by repeating changing the control parameter according to a predetermined rule until the temperature changes of the heater satisfy a predetermined determination condition while the test control is performed if the temperature changes of the heater do not satisfy the predetermined determination condition while the test control is performed.

5. The method of claim 4, wherein the control parameter includes an offset value or a proportional term, a derivative term, or an integral term of proportional-integral-differential control.

6. The method of claim 4, wherein the determination condition includes at least one of
a condition about a duration time of a temperature rise that is a time required to raise the temperature of the heater to a predetermined value, and
a condition about stability of the temperature of the heater when the test control is performed to maintain the temperature of the heater at a predetermined target temperature.

7. The method of claim 6, wherein
the condition about the duration time of the temperature rise is one second or shorter, and
the condition about the stability is that the temperature of the heater has a highest temperature and a lowest temperature between which a difference of 10° C. or less exists, when the test control is performed so that the temperature of the heater is maintained at the predetermined target temperature.

8. The method of claim 1, wherein
the test control includes
a first control of raising the temperature of the heater to a sealing temperature and maintaining the heater at the sealing temperature,
a second control of raising the temperature of the heater to a cutting temperature and maintaining the heater at the cutting temperature, and
a third control of raising the temperature of the heater to the sealing temperature and maintaining the heater at the sealing temperature, and then raising the temperature of the heater to the cutting temperature and maintaining the heater at the cutting temperature, and
the control parameter includes a first parameter for the first control, a second parameter for the second control, and a third parameter for the third control.

9. The method of claim 8, wherein
the sealing temperature is 100° C. or higher but 200° C. or lower, and
the cutting temperature is 200° C. or higher but 300° C. or lower.

10. A handpiece for a surgical system, the handpiece comprising:
a heater;
a first detection circuit configured to measure a voltage and a current to be supplied to the heater; and
a memory, wherein
the memory stores
detection characteristics representing a correlation between a sensor value of the first detection circuit and the at least one of the voltage value and the current value, the correlation being used for determination of the at least one of the voltage value and the current value being applied to the heater as obtained based on results measured using a second detection circuit having a higher detection accuracy than the first detection circuit, and
a control parameter which is to be used to control a temperature of the heater and is determined based on temperature changes of the heater while performing a test control that supplies electric power to the heater to control the temperature of the heater in a predetermined manner.

11. The handpiece of claim 10, wherein the control parameter is determined after obtaining the detection characteristics, and the temperature changes of the heater are obtained using the detection characteristics.

12. The handpiece of claim 10, wherein the detection characteristics of the first detection circuit are obtained by measuring, with each of the first detection circuit and the second detection circuit, a voltage and a current which are to be supplied to a load resistor having a resistance value of a maximum resistance value of the heater ±10Ω.

13. The handpiece of claim 10, wherein the control parameter is determined by repeating changing the control parameter according to a predetermined rule until the temperature changes of the heater satisfy a predetermined determination condition while the test control is performed if the temperature changes of the heater do not satisfy the predetermined determination condition while the test control is performed.

14. The handpiece of claim 13, wherein the control parameter includes an offset value or a proportional term, a derivative term, and/or an integral term of proportional-integral-differential control.

15. The handpiece of claim 13, wherein the determination condition includes at least one of
a condition about a duration time of a temperature rise that is a time required to raise the temperature of the heater to a predetermined value, and
a condition about stability of the temperature of the heater when the test control is performed to maintain the temperature of the heater at a predetermined target temperature.

16. The handpiece of claim 15, wherein
the condition about the duration time of the temperature rise is one second or shorter, and
the condition about the stability is that the temperature of the heater has a highest temperature and a lowest temperature, between which a difference of 10° C. or less exists, when the test control is performed so that the temperature of the heater is maintained at the predetermined target temperature.

17. The handpiece of claim 10, wherein
the test control includes
a first control of raising the temperature of the heater to a sealing temperature and maintaining the heater at the sealing temperature,
a second control of raising the temperature of the heater to a cutting temperature and maintaining the heater at the cutting temperature, and
a third control of raising the temperature of the heater to the sealing temperature and maintaining the heater at the sealing temperature, and then raising the temperature of the heater to the cutting temperature and maintaining the heater at the cutting temperature, and
the control parameter includes a first parameter for the first control, a second parameter for the second control, and a third parameter for the third control.

18. The handpiece of claim 17, wherein
the sealing temperature is 100° C. or higher but 200° C. or lower, and
the cutting temperature is 200° C. or higher but 300° C. or lower.

19. A surgical system comprising:
a handpiece having
a heater;
a first detection circuit configured to measure a voltage and a current to be supplied to the heater, and
a memory wherein the memory stores
detection characteristics representing a correlation between a sensor value of the first detection circuit and the at least one of the voltage value and the current value, the correlation being used for determination of the at least one of the voltage value and the current value being applied to the heater as obtained based on results measured using a second detection circuit having a higher detection accuracy than the first detection circuit, and
a control parameter which is to be used to control a temperature of the heater and is determined based on temperature changes of the heater while performing a test control that supplies electric power to the heater to control the temperature of the heater in a predetermined manner; and
a power source device configured to supply electric power to the heater of the handpiece, wherein
the power source device is configured to
obtain the detection characteristics and the control parameter from the memory of the handpiece, and
control the electric power which is to be supplied to the heater, based on a sensor value obtained by the first detection circuit, the detection characteristics, and the control parameter.
